(19) United States
(12) Patent Application Publication
(10) Pub. No.: US 2018/0085512 A1
(43) Pub. Date: Mar. 29, 2018

(54) FILTERING A PRESSURE SIGNAL FROM A PRESSURE SENSOR IN A BLOOD PROCESSING APPARATUS

(71) Applicant: GAMBRO LUNDIA AB, Lund (SE)

(72) Inventors: Bo OLDE, Lund (SE); Jan STERNBY, Lund (SE); Kristian SOLEM, Trelleborg (SE)

(21) Appl. No.: 15/573,223

(22) PCT Filed: Jun. 3, 2016
(86) PCT No.: PCT/EP2016/062619

§ 371 Date: Nov. 10, 2017

(30) Foreign Application Priority Data
Jun. 25, 2015 (SE) 1550882-3

(51) Int. Cl. A61M 1/36; A61B 5/021; A61B 5/00

(52) U.S. Cl. CPC A61M 1/3639; A61B 5/02108; A61M 2230/30; A61B 5/725; A61M 2205/3331; A61B 5/7246

(57) ABSTRACT

A signal filtering device acquires a pressure signal from a pressure sensor in a blood processing apparatus having a known disturbance generator that generates a disturbance in the pressure signal. The signal filtering device obtains (401) a current set of principal components representing the disturbance. The respective principal component in the current set is generated by Principal Component Analysis, PCA, of a plurality of characteristic waveforms representing the disturbance. The signal filtering device further operates to detect (403) presence in the pressure signal of a current disturbance that originates from the disturbance generator, computes (404) a scaling factor for the respective principal component with respect to the current disturbance, and subtracts (405) the respective principal component, scaled in magnitude by the respective scaling factor, from the pressure signal. The signal filtering device may be associated with or included in the blood processing apparatus.

### TECHNICAL FIELD

The present invention relates to processing of a pressure signal obtained from a pressure sensor in a blood processing apparatus, and in particular to filtering of the pressure signal for suppression of disturbances originating from the blood processing apparatus.

### BACKGROUND ART

In extracorporeal blood processing, blood is taken out of a human or animal subject, processed (e.g. treated) and then reintroduced into the subject by means of an extracorporeal blood flow circuit (“EC blood circuit”) which is part of a system for blood processing. Generally, the blood is circulated through the EC blood circuit by a blood pump. In certain types of extracorporeal blood processing, the EC blood circuit includes an access device for blood withdrawal (e.g. a so-called arterial needle) and an access device for blood reintroduction (e.g. a so-called venous needle), which are inserted into a dedicated blood vessel access (e.g. fistula or graft) on the subject. Such extracorporeal blood processing includes hemodialysis, hemodiafiltration, hemo-filtration, plasmapheresis, etc.

In extracorporeal blood processing, it is vital to minimize the risk for malfunctions in the EC blood circuit, since these may lead to a potentially life-threatening condition of the subject. Serious conditions may e.g. arise if the EC blood circuit is disrupted downstream of the blood pump, e.g. by a VND event (VND—Venous Needle Dislodgement), in which the venous needle comes loose from the blood vessel access. Such a disruption may cause the subject to be drained of blood within minutes.

VND may be detected during blood processing based on a venous pressure signal from a pressure sensor (“venous pressure sensor”) on the downstream side of the blood pump in the EC circuit. Such VND monitoring may be carried out by identifying changes in the pressure level of the venous pressure signal, optionally in relation to changes in the pressure level of an arterial pressure signal from a pressure sensor (“arterial pressure sensor”) located upstream of the blood pump, e.g. as described in U.S. Pat. No. 6,221,040 or US2011/0034814. Alternatively, VND monitoring may be carried by detecting an absence of heart pulses in the venous pressure signal. The heart pulses represent pressure pulses produced by a patient's heart and transmitted from the patient's circulatory system to the venous pressure sensor via the blood vessel access and the venous needle. An absence of heart pulses in the pressure signal is taken as an indication of a possible VND event. Such techniques are e.g. disclosed in WO97/10013, US2005/0010118, WO2009/156174 and US2010/0234786. As an alternative, WO2010/149726 proposes VND monitoring based on detection of physiological pulses other than heart pulses in the venous pressure signal, e.g. from reflexes, voluntary muscle contractions, non-voluntary muscle contractions, the breathing system, the autonomous system for blood pressure regulation or the autonomous system for body temperature regulation.

It has also been proposed to monitor and analyze the behavior of physiological pressure generators such as the heart or respiratory system in the subject connected to the EC blood circuit, by detecting physiological pulses in the venous or arterial pressure signal. Various implementations are described in WO2010/149726, WO2011/080189, WO2011/080190, WO2011/080191 and WO2011/080194. As a further implementation, WO2011/080188 proposes a technique for identifying and signaling a reverse placement of the venous and arterial needles in the vascular access by detecting and analyzing physiological pulses in the venous and/or arterial pressure signal.

In order to provide a consistent and reliable monitoring, it is important to ensure that the pressure signal is substantially free from disturbances that may interfere with the detection of pressure changes or physiological pulses, as the case may be. The disturbances may be suppressed or removed by conventional filtering of the pressure signal. The task of filtering may be rendered difficult if the disturbances overlap in frequency with the pressure changes or physiological pulses to be monitored, since the filtering should not significantly interfere with the detection of pressure changes or physiological pulses.

For example, it is known that strong repetitive pulsations from the blood pump (“pump pulses”) may be present in the pressure signal at a rate similar to the heart pulsations. In this respect, WO2009/156175 proposes techniques for filtering a pressure signal in the time domain for the purpose of eliminating (or suppressing) the pump pulses while retaining the physiological pulses. These techniques involve estimating the shape of the pump pulses, by obtaining a “predicted signal profile”, at the relevant operating condition of the EC blood circuit and by subtracting the predicted signal profile from the pressure signal. In one implementation, a library of predicted signal profiles is recorded in a reference measurement before treatment, e.g. during a priming phase or during a simulated treatment, at a plurality of different operating conditions of the EC blood circuit. Each predicted signal profile is generated as an average of pump pulses recorded by a pressure sensor in the EC blood circuit. In another implementation, the library of predicted signal profiles is generated by simulations using a mathematical model of the EC blood circuit. Based on the current operating condition of the EC blood circuit, a predicted signal profile may be selected from the library and used for eliminating the pump pulses.

There is a continued need to achieve an improved filtering technique, in terms of one or more of the following: ability to handle overlap in frequency and/or time between disturbances and physiological pulses, complexity of the filtering technique, ability to generate the filtered signal in real time, processing efficiency and memory usage during filtering, accuracy of the filtered signal, and robustness of the filtering technique.

The pressure sensors in the EC blood circuit may also be responsive to pressure variations with other origin than the blood pump, e.g. switching of valves, operation of other pumps, operation of balancing chambers, etc. These pressure variations may be manifested as distinct pulses in the pressure signal, which may be removed by conventional filtering techniques. However, the present Applicant has found that the distinct pulses with certain origin exhibit variations in shape and/or magnitude and/or duration over time and that these variations make it difficult to apply existing filtering techniques. For example, the technique of subtracting a predicted signal profile that represents the disturbance as proposed in WO2009/156175 presumes that the shape of the disturbance is consistent between different occurrences of the disturbance in the pressure signal. Even small variations in the shape of the disturbance may result in residuals of the disturbance in the pressure signal, since the disturbance is filtered by subtraction of a predicted signal profile for the disturbance. The residuals may interfere with the monitoring, especially if the disturbance is a dominant feature in the pressure signal, e.g. significantly stronger magnitude than the pressure changes or physiological pulses to be monitored.

### SUMMARY

It is an objective of the invention to at least partly overcome one or more of the above-identified limitations of the prior art.

Another objective is to provide an improved technique for filtering a pressure signal from a pressure sensor in a blood processing apparatus.

A still further objective is to provide such a filtering technique that is capable of meeting one or more of the above-mentioned needs.

A further objective is to provide a filtering technique capable of adapting to variations in the appearance of a disturbance with a known origin in the pressure signal.

One or more of these objectives, as well as further objectives that may appear from the description below, are at least partly achieved by signal filtering devices, a method of filtering, a computer-readable medium and a blood processing apparatus according to the independent claims, embodiments thereof being defined by the dependent claims.

A first aspect of the invention is a signal filtering device comprising an input for receiving a pressure signal from a pressure sensor, which is arranged in a blood processing apparatus and is responsive to pressure variations in blood that is pumped in an extracorporeal blood circuit for passage through a blood processing unit, at least part of the pressure variations originating from a known disturbance generator in the blood processing apparatus and resulting in a disturbance in the pressure signal. The signal filtering device further comprises a signal processor connected to the input and being configured to, in connection with a current session of the blood processing apparatus: obtain a set of principal components representing the disturbance, the respective principal component in the set of principal components being generated by Principal Component Analysis, PCA, of a plurality of characteristic waveforms representing the disturbance; detect presence in the pressure signal of a current disturbance that originates from the known disturbance generator; compute a scaling factor for the respective principal component with respect to the current disturbance; and generate a filtered signal by subtracting the respective principal component, scaled in magnitude by the respective scaling factor, from the pressure signal.

Principal component analysis, PCA, is a statistical procedure that is mostly used as a tool in exploratory data analysis and for making predictive models. Depending on the field of application, PCA is also named the discrete Karhunen-Loéve Transform (KLT), the Hotelling Transform, Proper Orthogonal Decomposition (POD), Eckart-Young Theorem, Empirical Orthogonal Functions (EOF), and Spectral Decomposition. A common feature of PCA algorithms is that a covariance matrix (or equivalently, a correlation matrix) is estimated for a data set and processed for computation of eigenvectors and possibly eigenvalues, so that principal components and possibly variances can be defined based on the eigenvectors and the eigenvalues, respectively. The principal components are thus uncorrelated and take the form of basis functions for the data set. The variances, if computed, represent the variability of the data set along the respective principal component. PCA algorithms produce a first principal component that has the largest possible variance and thus accounts for as much as possible of the variability within the data set, and may produce succeeding principal components that each has the highest variance possible under the constraint that it is orthogonal to (i.e., uncorrelated with) the preceding components.

The first aspect is based on the insight that principal components, when generated by PCA processing of a plurality of recorded occurrences of the disturbance, are relatively unaffected if some recorded occurrences should deviate significantly in appearance (shape, magnitude, duration) from other recorded occurrences. These significantly deviating occurrences form “outliers” in the data set that is processed by PCA. Due to the inherent property of PCA, these outliers will have a minor impact on the resulting principal components, e.g. compared to a signal profile computed by averaging the recorded occurrences. Furthermore, if using two or more principal components, the first aspect has the ability of adapting to small variations in appearance between different occurrences of the disturbance in the pressure signal. As noted above, the principal components form basis functions for the recorded occurrences. This means that a subtraction of two or more principal components that are scaled in magnitude by the respective scaling factor corresponds to a subtraction of a template or signal profile that is adjusted to the appearance of the individual disturbance in the pressure signal. Thus, the inventive filtering technique is robust and has the ability of producing a filtered signal without large residuals when there is variability in the appearance of the disturbance.

The inventive filtering technique is suitably applied to suppress disturbances in a pressure signal that is acquired during a session of the blood processing apparatus, i.e. when the blood processing apparatus is connected a patient and operated to process the blood of the patient.

While the underlying mathematical theory of PCA is complex, there are established and processing-efficient PCA algorithms that may be efficiently implemented in either hardware or software, or a combination of thereof, to compute the principal components. The scaling factors may be obtained by simple and standard operations, such as by computation of scalar products (dot products) between two vectors. Likewise, the rescaling and subtraction of the respective principal component may be implemented by simple and standard operations. Thus, the inventive filtering technique may be implemented with a low complexity and high processing efficiency, and thereby with the ability of generating a filtered signal in real time. Furthermore, to the extent that the current set of the principal components are stored in memory, the memory usage is small.

As understood from embodiments presented further below, the processing by PCA may but need not be performed by the signal filtering device and may but need not operate on characteristic waveforms extracted from the pressure signal to be filtered. In all of the following examples, the processing by PCA involves producing a set of eigenvectors for an estimated covariance matrix with estimated covariance values for the characteristic waveforms, whereupon one or more principal components are given by a respective eigenvector in the set of eigenvectors.

In one example, the processing by PCA is performed externally of the signal processing device, by a separate device. The separate device pre-computes a set of principal components, which are transferred to and stored in a memory which is accessible to the signal filtering device. The pre-computed set of principal components may then be retrieved from the memory by the signal filtering device and used as the current set of principal components.

In another example, the processing by PCA is performed by the signal processing device, e.g. based on characteristic waveforms that are extracted from the pressure signal to be filtered, to produce the current set of principal components.

In a further example, the signal filtering device may be operable to generate the current set of principal components by modifying a set of principal components stored in the memory of the signal filtering device, e.g. by computing a weighted combination of each stored principal component with a respective new principal component generated by PCA in the signal filtering device. The stored set of principal components may be pre-computed by PCA in a separate device or be previously computed by PCA in the signal filtering device.

It is thus realized that the current set of principal components is generated by processing a plurality of characteristic waveforms by PCA. The processing may be consolidated such that all waveforms are processed at one time to generate the current set of principal components. Alternatively, the processing may be distributed, such that the current set of principal components is formed by combining sets of principal components generated by PCA on different subsets of the plurality of characteristic waveforms. For example, one subset may be processed on the separate device, and another subset may be processed on the signal filtering device, and/or different subsets may be processed by the signal filtering device at different time points.

In one embodiment with PCA processing in the signal filtering device, the signal processor is configured to: extract all or part of the characteristic waveforms from the pressure signal, each characteristic waveform representing a respective occurrence of the disturbance in the pressure signal; process said all or part of the characteristic waveforms by PCA so as to compute a set of principal components for said all or part of the characteristic waveforms; define a selected set of principal components among the set of principal components; and generate the current set of principal components as a function of the selected set of principal components. In one implementation, the signal processor is further configured to, when processing said all or part of the characteristic waveforms by PCA, compute a corresponding set of variances for the set of principal components, and to select, based on the set of variances, the principal components for the selected set of principal components. For example, the signal processor may select one or more of the principal components with the largest variance.

In one embodiment, the signal processor is configured to, when processing said all or part of the characteristic waveforms by PCA, to compute a set of eigenvectors for an estimated covariance matrix with estimated covariance values for said all or part of the characteristic waveforms, the set of principal components being given by the set of eigenvectors. The estimated covariance matrix may be given by ƒ(X^{T}X), wherein X is a matrix with said all or part of the characteristic waveforms arranged as rows or columns, X^{T} is a transpose of the matrix X, and ƒ is a linear function.

In one embodiment, the signal processor is further configured, when processing said all or part of the characteristic waveforms by PCA, to: compute the estimated covariance values; populate the estimated covariance matrix by the estimated covariance values; and process the estimated covariance matrix for computation of the set of eigenvectors.

In one embodiment, the signal processor is configured to extract said all or part of the characteristic waveforms from the pressure signal during the current session, such as during a startup phase of the current session.

In one embodiment, the signal processor is configured to use the selected set of principal components as the current set of principal components.

In an alternative embodiment, the signal processor is configured to: retrieve a stored set of principal components from an electronic memory associated with the signal filtering device; and obtain the current set of principal components as a combination of the stored set of principal components and the selected set of principal components. The signal processor may be further configured to: store the current set of principal components in the electronic memory so as to replace the stored set of principal components in the electronic memory. The stored set of principal components may be pre-computed externally of the signal filtering device, or may be a previously computed current set of principal components.

In one embodiment, the signal processor is configured to obtain the current set of principal components by retrieving a pre-computed set of principal components from an electronic memory associated with the signal filtering device, the pre-computed set of principal components being generated externally of the signal filtering device. The pre-computed set of principal components may be specific to one of: an apparatus type of the blood processing apparatus, the blood processing apparatus, a patient connected to the blood processing apparatus, and a combination of the blood processing apparatus and the patient.

In one embodiment, the signal processor is operable to obtain the current set of principal components to be specific to one of: an apparatus type of the blood processing apparatus, the blood processing apparatus, a patient connected to the blood processing apparatus, a combination of the blood processing apparatus and the patient, and the current session.

In one embodiment, the signal processor is configured to compute the scaling factor as a scalar product of the respective principal component in the current set of principal components and a signal vector representing the current disturbance in the pressure signal.

In one embodiment, the signal processor is configured to detect the presence of the current disturbance by one or more of: processing the pressure signal for detection of a dominant feature of the disturbance; cross-correlating a selected principal component in the current set of principal components with the pressure signal to generate a plurality of correlation values, and processing the plurality of correlation values for detection of the current disturbance; and receiving, via the input, a reference signal which is indicative of the operation of the known disturbance generator, and detecting, based on the reference signal, an activation or deactivation of the known disturbance generator that results in the current disturbance.

In one embodiment, the signal processor is further configured to: determine a time point of the current disturbance in the pressure signal; and align the respective principal component with respect to the time point when subtracting the respective principal component from the pressure signal.

In one embodiment, the signal processor is further configured to: generate a filtered signal, and process the filtered signal for detection of pulsations originating from one or more physiological pulse generators in the patient.

A second aspect of the invention is a signal filtering device comprising: means for receiving a pressure signal from a pressure sensor, which is arranged in a blood processing apparatus and is responsive to pressure variations in blood that is pumped in an extracorporeal blood circuit for passage through a blood processing unit, at least part of the pressure variations originating from a known disturbance generator in the blood processing apparatus and resulting in a disturbance in the pressure signal; means for providing a set of principal components representing the disturbance, the respective principal component in the set of principal components being generated by Principal Component Analysis, PCA, of a plurality of characteristic waveforms representing the disturbance in the pressure signal; means for detecting presence in the pressure signal of a current disturbance that originates from the known disturbance generator; means for computing a scaling factor for the respective principal component with respect to the current disturbance; and means for generating a filtered signal by subtracting the respective principal component, scaled in magnitude by the respective scaling factor, from the pressure signal.

A third aspect of the invention is a method of filtering, comprising: acquiring a pressure signal originating from a pressure sensor, which is arranged in a blood processing apparatus and is responsive to pressure variations in blood that is pumped in an extracorporeal blood circuit for passage through a blood processing unit, at least part of the pressure variations originating from a known disturbance generator in the blood processing apparatus and resulting in a disturbance in the pressure signal; obtaining a set of principal components representing the disturbance, the respective principal component in the set of principal components being generated by Principal Component Analysis, PCA, of a plurality of characteristic waveforms representing the disturbance in the pressure signal; detecting presence in the pressure signal of a current disturbance that originates from the known disturbance generator; computing a scaling factor for the respective principal component with respect to the current disturbance; and generating a filtered signal by subtracting the respective principal component, scaled in magnitude by the respective scaling factor, from the pressure signal.

A fourth aspect of the invention is a computer-readable medium comprising computer instructions which, when executed by a processor, cause the processor to perform the method of the third aspect.

A fifth aspect of the invention is a blood processing apparatus, comprising an extracorporeal blood circuit and a pressure sensor, the pressure sensor being responsive to pressure variations in blood that is pumped in the extracorporeal blood circuit for passage through a blood processing unit, said blood processing apparatus further comprising the signal filtering device of the first or second aspects which is connected to receive the pressure signal from the pressure sensor.

Any one of the above-identified embodiments of the first aspect may be adapted and implemented as an embodiment of the second to fifth aspects.

Still other objectives, features, aspects and advantages of the present invention will appear from the following detailed description, from the attached claims as well as from the drawings.

### BRIEF DESCRIPTION OF DRAWINGS

Embodiments of the invention will now be described in more detail with reference to the accompanying schematic drawings.

FIG. 1 a schematic diagram of an extracorporeal blood processing apparatus attached to a human subject.

FIG. 2 is a flow chart for a method of generating principal components representing a disturbance in a pressure signal.

FIG. 3A is a plot of disturbances, and FIGS. 3B-3C illustrate first and second principal components computed for the disturbances in FIG. 3A.

FIG. 4 is a flow chart of a filtering method according to an embodiment.

FIG. 5A illustrates alignment of a first principal component with a pressure signal before subtraction, and FIG. 5B illustrates the pressure signal before and after subtraction.

FIG. 6A is a plot of disturbances, FIG. 6B is a plot of signals generated by subtraction of the first principal component in FIG. 3B from the disturbances in FIG. 6A, and FIG. 6C is a plot of signals generated by subtraction of the second principal component in FIG. 3C from the signal in FIG. 6B.

FIG. 7 is a block diagram of a structure for implementing the method in FIG. 4.

FIG. 8 is a block diagram of an alternative structure for implementing the method in FIG. 4.

FIGS. 9A-9B illustrate first and second principal components, respectively, generated for individual treatment sessions of an apparatus, together with a corresponding principal component generated for all individual sessions.

FIGS. 10A-10B illustrate, for two individual apparatuses, first principal components generated for individual treatment sessions of the respective apparatus, together with a corresponding principal component generated for all of the individual sessions, and FIG. 10C illustrates the first principal components generated for individual apparatuses, together with a corresponding principal component generated for all of the apparatuses.

### DETAILED DESCRIPTION OF EXAMPLE EMBODIMENTS

Throughout the description, the same reference numerals are used to identify corresponding elements.

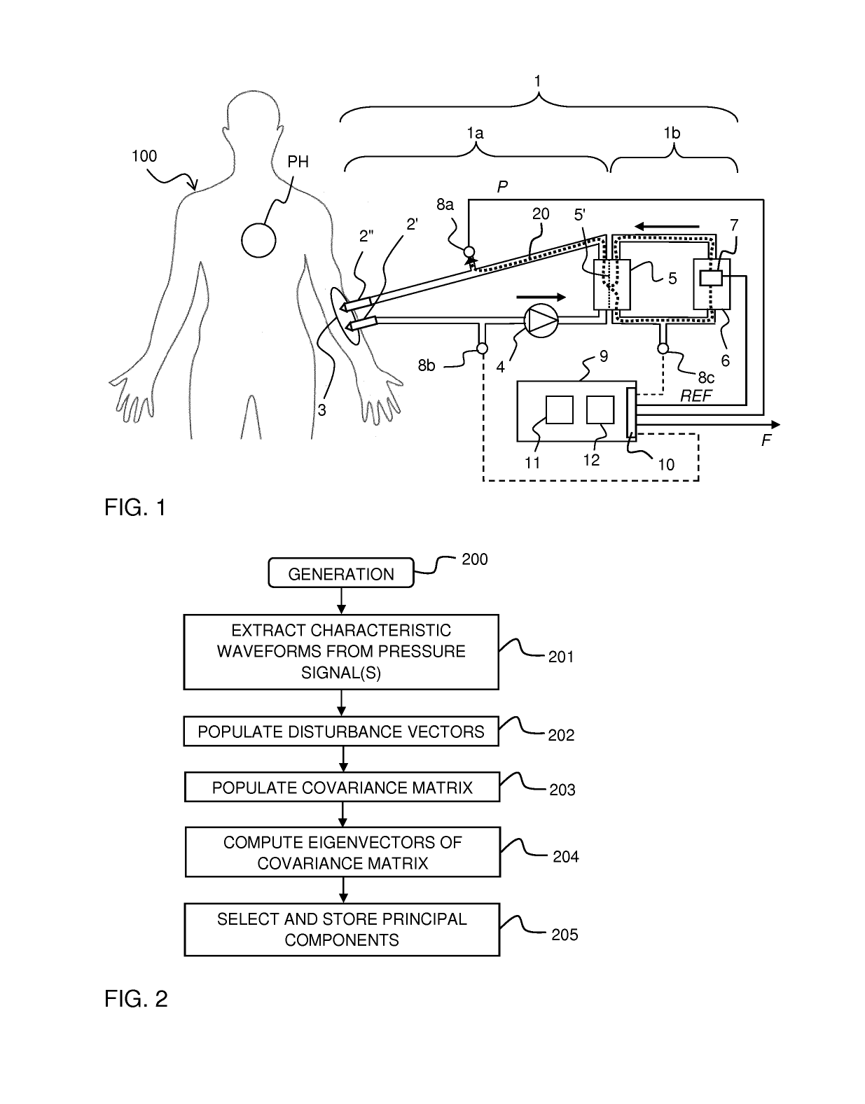

Embodiments of the invention will be exemplified with reference to an apparatus 1 for blood treatment, which is schematically depicted in FIG. 1. In the following example, the apparatus is assumed to be a dialysis system which is formed by a blood line set attached to a dialysis machine or monitor, as is well known in the art. FIG. 1 illustrates a human subject or patient 100 which is connected to an extracorporeal blood flow circuit 1a by way of access devices 2′, 2″ inserted into a dedicated vascular access 3 (also known as “blood vessel access”) on the patient. The extracorporeal blood flow circuit 1a (denoted “EC circuit” in the following) is configured to communicate blood to and from the cardiovascular system of the patient 100. In the illustrated example, a blood pump 4 draws blood from the vascular access 3 via an access device 2′ for blood withdrawal and pumps the blood through a blood treatment unit 5 and back to the vascular access 3 via an access device 2″ for blood return. Thus, when both access devices 2′, 2″ are connected to the vascular access 3, the EC circuit 1a defines a blood path that starts and ends at the vascular access 3. The EC circuit 1a may be seen to comprise a “venous side” which is the part of the blood path located downstream of the blood pump 4, and an “arterial side” which is the part of the blood path located upstream of the blood pump 4. The blood pump 4 may be of any type, e.g. a rotary peristaltic pump, a linear peristaltic pump, a diaphragm pump, or a centrifugal pump.

The blood treatment unit 5 may be any type of blood filtration device, such as a coil dialyzer, a parallel plate dialyzer, a hollow fiber dialyzer, etc. For simplicity, the blood treatment unit 5 is denoted “dialyzer” in the following. The dialyzer 5 has a blood side and a treatment fluid side separated by a semipermeable membrane 5′. The blood side is connected as part of the EC circuit 1a, and the treatment fluid side is connected as part of a supply system for treatment fluid 1b (denoted “TF circuit” in the following). The TF circuit 1b is arranged to pump a treatment fluid through the treatment fluid side of the dialyzer 5, whereby solutes are transported over the membrane 5′ due to a concentration gradient and/or ultrafiltrate is transported over the membrane 5′ due to a pressure gradient. The skilled person understands that the TF circuit 1b may include a plurality of functional components such as a source of fresh treatment fluid, a receptacle/drain for spent treatment fluid, one or more pumps, balancing chambers, valves, heaters, conductivity sensors, degassing chambers, etc. For simplicity, these components are collectively represented by a generic box 6 in FIG. 1.

It is understood that the EC circuit 1a and the TF circuit 1b form part of the above-mentioned apparatus 1 for blood treatment. A control unit (not shown) in the apparatus 1 controls and synchronizes the operation of, e.g., the blood pump 4 and the components 6, as well as further components such as pumps, sensors, valves, a user interface, etc.

The apparatus 1 is operated in individual treatment sessions. As used herein a treatment session (“session”) refers to an isolated event, in which the apparatus 1 is connected to the patient 100 and then operated to process the blood of the patient 100, whereupon the apparatus 1 is disconnected from the patient 100.

The EC circuit 1a includes a pressure sensor or transducer 8a (denoted “venous pressure sensor” or “venous sensor”) on the venous side of the EC circuit 1a, downstream of the dialyzer 5, a pressure sensor or transducer 8b (denoted “arterial pressure sensor” or “arterial sensor”) on the arterial side of the EC circuit 1a. The venous and arterial sensors 8a, 8b provide a respective time-varying signal that represents the pressure in the blood on the venous side (“venous signal”) and the arterial side (“arterial signal”), respectively. In the example of FIG. 1, a pressure sensor or transducer 8c (denoted “TF pressure sensor” or “TF sensor”) is also arranged in the TF circuit 1b to provide a time-varying signal that represents the pressure in the treatment fluid. The TF sensor 8c may have any placement in the TF circuit 1b, e.g. downstream (as in FIG. 1) or upstream of the dialyzer 5.

The pressure sensors 8a-8c may be of any type, e.g. operating by resistive, capacitive, inductive, magnetic, acoustic or optical sensing, and using one or more diaphragms, bellows, Bourdon tubes, piezo-electrical components, semiconductor components, strain gauges, resonant wires, accelerometers, etc. For example, each of the pressure sensors 8a-8c may be implemented as a conventional pressure sensor, a bioimpedance sensor, or a photoplethysmography (PPG) sensor.

A filtering device 9 is connected to at least the venous pressure sensor 8a by a data line to acquire and process the venous signal, which is a time-varying electric pressure signal designated by P in FIG. 1. The device 9 may be included as part of the apparatus 1 for blood treatment and may be connected to or be part of the above-mentioned control unit. Alternatively, the device 9 is separate from the apparatus 1. The dashed data lines from the arterial and TF sensors 8b, 8c to the device 9 indicate that the use of signals from these sensors is optional, as will be described further below.

Specifically, the filtering device 9 comprises an input or signal interface 10 adapted to receive at least the pressure signal P during a treatment session, and processing circuitry 11, 12 for processing the pressure signal P to suppress disturbances that originate from one or more disturbance generators 7 in the apparatus 1. The processing results in a filtered signal F, which may be output via the signal interface 10. Embodiments of the invention may e.g. be implemented by software instructions that are supplied on a computer-readable medium for execution by a processor 11 in conjunction with an electronic memory 12 in the device 9.

In the following examples, the filtering device 9 is operable to suppress disturbances representing pressure waves that enter the EC circuit 1a via the dialyzer 5, propagate in the blood to the sensor 8a. As used herein, a “pressure wave” is a mechanical wave that travels or propagates through a material or substance. The sensor 8a, which is in direct or indirect hydraulic contact with the blood, is responsive to these pressure waves and generates a representative disturbance in the pressure signal. A “disturbance” is thus a set of data samples that define a local change in signal magnitude within a pressure signal. The disturbance may but need not be in the form of a pulse.

FIG. 1 illustrates by dotted lines a propagation path 20 for pressure waves that are generated by a disturbance generator 7 included in the apparatus 1, specifically in the TF circuit 1b. As seen, the pressure waves propagate in the treatment fluid to the dialyzer 5, where they pass the membrane 5′ and propagate in the blood to the venous sensor 8a and result in disturbances in the pressure signal P. The disturbance generator 7 may be a single component or device, or a combination of devices that operate in conjunction to generate one or more pressure waves that form a coherent disturbance in the pressure signal P. In the example of a dialysis machine, the disturbance may e.g. originate from changes in the flow rate of treatment fluid, redirection or restriction or occlusion of fluid flow by switching of valves, degassing of the treatment fluid, UF calibration, a change of operating mode of the dialysis machine, etc.

Generally, the filtering device 9 operates to suppress disturbances in the pressure signal P by subtracting a signal template or waveform that is representative of the respective disturbance. The signal template is formed by a set of basis functions that has been generated for the disturbance to be suppressed. It is to be understood that if the pressure signal P contains disturbances from different disturbance generators 7, each disturbance is suppressed by subtraction of a respective signal template given by a specific set of basis functions associated with respective disturbance. The basis functions are principal components obtained by processing a plurality of occurrences of the disturbance by Principal Component Analysis (PCA). Hence, the filtering technique is referred to as “PCA filtering” herein.

The PCA filtering is especially suited for suppression of disturbances that exhibit certain variability in shape between occurrences in the pressure signal P. Such variability is not uncommon for disturbances than occur only intermittently in the pressure signal P, and in particular if the disturbances are well-separated in time, e.g. by seconds, tens of seconds, minutes, even or tens of minutes. The disturbances may or may not occur periodically, i.e. with an essentially invariant time separation.

It should be emphasized that the disturbance generator 7 need not be located in the TF circuit 1b but could be located elsewhere in the apparatus 1 so as to generate pressure waves that enter the TF circuit 1b and then propagate into the EC circuit 1a via the dialyzer 5. It is also conceivable that the pressure waves enter the EC circuit 1a on another pathway than via the dialyzer 5. For example, pressure waves may enter the EC circuit 1a from a supply line for substitution fluid connected to the EC circuit 1a, from a drip chamber in the EC circuit 1a, from a connection to a heparin pump, etc. In another alternative, the disturbance generator 7 is located in the EC circuit 1a. For example, the blood pump 4 is known to generate strong pressure waves that form periodic pulses (“pump pulses”) in the pressure signal P. The shape of the pump pulses is typically consistent over time. While the pump pulses may be suppressed by the PCA filtering, the present disclosure presumes that the filtering device 9 applies some other, well-known filtering technique for suppressing the pump pulses.

The filtered signal F may be further processed, by the device 9 or a separate device (not shown), for any type of monitoring purpose, e.g. as described in the Background section. For example, if the filtered signal F is generated to represent the momentary average pressure at the sensor 8a, i.e. the “DC level” of the venous signal P, the filtered signal F may be monitored for detection of pressure changes corresponding to a disruption of the EC circuit 1a downstream of the blood pump 4, known as a VND event. Alternatively, the filtered signal F may be generated to include pressure pulsations (“patient pulses”) that originate from a physiological pulse generator PH in the patient 100, such as reflexes, voluntary muscle contractions, non-voluntary muscle contractions, the heart, the breathing system, the autonomous system for blood pressure regulation and the autonomous system for body temperature regulation, or from a mechanical device (not shown) which is attached to the patient 100 or a support for the patient 10, e.g. a bed, and which shakes, vibrates or presses so as to generate pressure waves that propagate in the cardiovascular system of the patient 100, via the access device 2″ to the pressure sensor 8a so as to form the patient pulses in the pressure signal P.

It should also be understood that the PCA filtering additionally or alternatively may be used for filtering pressure signals from other sensors in the apparatus 1, such as the TF signal from sensor 8c or the arterial signal from sensor 8b, whereupon the resulting filtered signal may be used for any monitoring purpose.

The PCA filtering involves two main parts: generating principal components for a disturbance by PCA processing, and filtering the pressure signal P by use of one or more of the principal components.

PCA is a statistical procedure that uses an orthogonal linear transformation to convert a set of observations of correlated variables into a set of values of linearly uncorrelated variables called “principal components”. The number of principal components is less than or equal to the number of original variables. This transformation is defined in such a way that the first principal component has the largest possible variance, i.e. accounts for as much of the variability in the data as possible, and each succeeding component in turn has the highest variance possible under the constraint that it is orthogonal to (i.e., uncorrelated with) the preceding components. Thus, PCA processing results in a number of principal components for the set of observations, and may also result in a variance for each principal component.

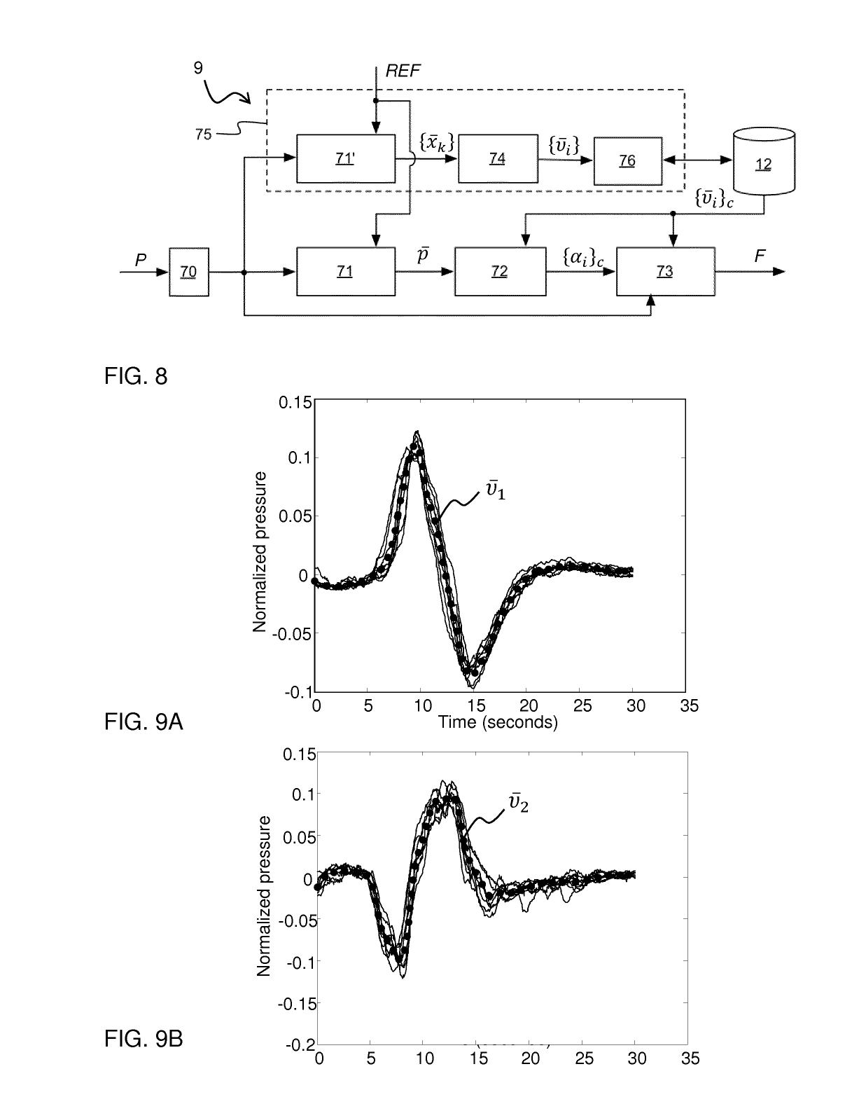

FIG. 2 illustrates a process 200 for generating principal components for a disturbance in the example environment of FIG. 1. The process 200 may but need not be executed by the filtering device 9. The process operates on a plurality of characteristic waveforms or recordings of the disturbance. Each waveform corresponds to an occurrence of the disturbance and may be extracted from the pressure signal P to be filtered. As will be explained in more detail with reference to FIGS. 7-8, it is conceivable that all or some of the waveforms may be extracted from a pressure signal acquired from the venous sensor 8a in another apparatus of the same type. Thus, depending on implementation, step 201 may involve identifying and extracting at least some of the waveforms in real time from a pressure signal, or retrieving previously extracted waveforms from an electronic memory. The disturbance may be identified in the pressure signal P based on an external reference signal that is directly or indirectly associated with the activation or deactivation of the disturbance generator (cf. REF in FIG. 1). Alternatively, the disturbance may be identified by processing the pressure signal P for detection of one or more dominating features of the disturbance, e.g. one or more peaks or one or more slopes (flanks) that define a pressure change of sufficient magnitude and/or duration in the pressure signal P.

All waveforms that are input to the generation process 200 consist of an equal number of data samples and represent the disturbance in the same time scale. Further, the location of the disturbance is the same in all waveforms. In other words, the disturbances that are represented by the waveforms are aligned by a common reference time point in each disturbance. The reference time point may be given by a feature of the disturbance (maximum/minimum values, maximum slope, etc), or a time point indicated by the above-mentioned reference signal.

Step 202 populates a set of disturbance vectors such that each disturbance vector corresponds to a respective waveform. In the following, the disturbance vectors are designated by x̅ₖ, k=1, . . . , n. Each disturbance vector contains m signal values:

$${\overline{x}}_{k} = [ \begin{matrix}{x}_{k} ( 1 ) \\ {x}_{k} ( 2 ) \\ ⋮ \\ {x}_{k} ( m )\end{matrix} ] .$$

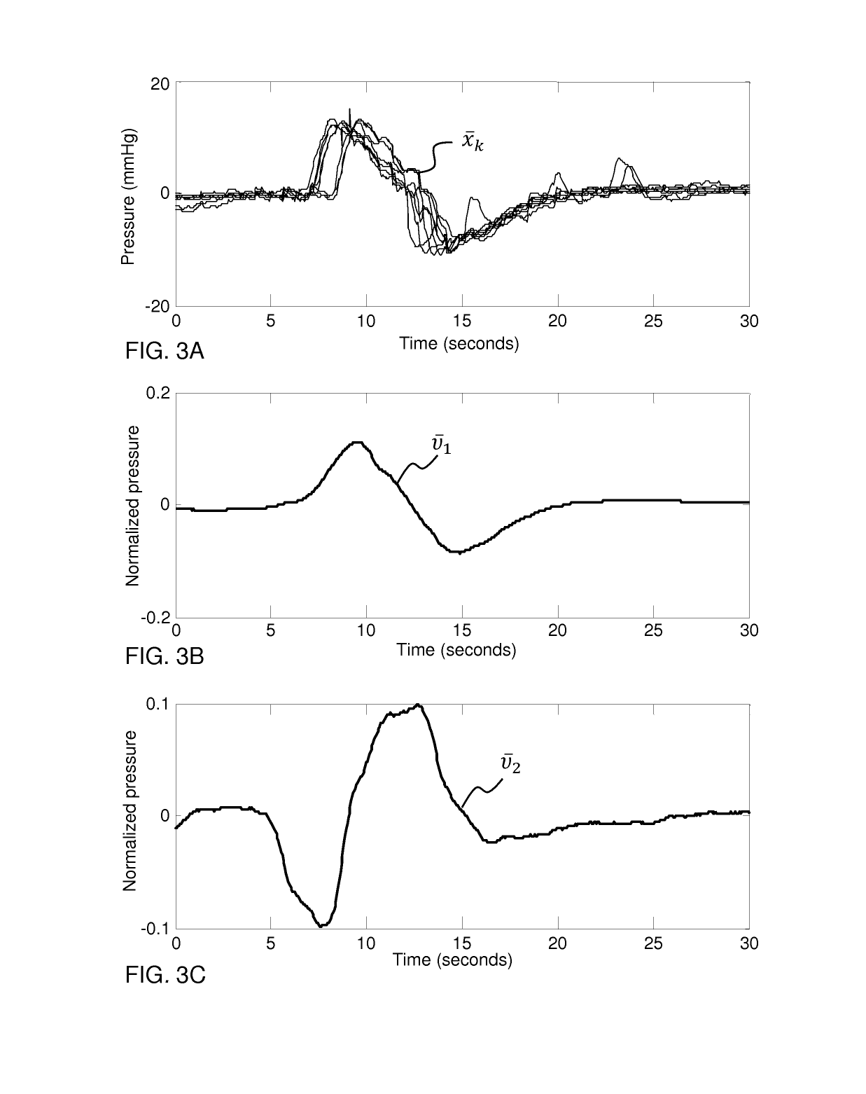

As an example, FIG. 3A illustrates eight disturbance vectors x̅ₖ representing a corresponding number of occurrences of a disturbance from the disturbance generator 7 in the pressure signal P. In this and other examples given herein, each disturbance vector x̅ₖ represents a time window of 30 seconds, corresponding to 300 pressure values (data samples) at a sampling rate of 10 Hz.

As used herein, a variable enclosed in { } indicates a set of values for the variable. For example, the set of disturbance vectors is designated by {x̅ₖ}.

Steps 203-204 then processes the set {x̅ₖ} by PCA to compute principal components, each designated by ν̅ᵢ, and a variance for each principal component. Theoretically, PCA involves applying an orthogonal linear transformation to the disturbance vectors that are at least partially correlated, so as to generate transformed disturbance vectors that are uncorrelated. The thus-transformed disturbance vectors form principal components for the disturbance vectors.

It can be shown that finding the transformed disturbance vectors is equivalent to finding the eigenvectors of the covariance matrix for the set {x̅ₖ}. The covariance matrix may be estimated for the set of disturbance vectors by forming a data matrix X in which the disturbance vectors x̅ₖ are arranged as rows:

$$X = [ \begin{matrix}{\overline{x}}_{1}^{T} \\ {\overline{x}}_{2}^{T} \\ ⋮ \\ {\overline{x}}_{n}^{T}\end{matrix} ]$$

and by evaluating the matrix operation X^{T}·X, where superscript T indicates a transpose. The estimated covariance matrix C is given by ƒ(X^{T}·X), where ƒ is any suitable linear function. For example, the function ƒ may be designed to normalize X^{T}·X in proportion by the number of disturbance vectors, e.g. through a division by n or n−1. The data matrix X has size n×m (number of rows times number of columns), and the estimated covariance matrix C contains m×m estimated covariance values. It can be noted that X^{T}·X corresponds to the element-wise sum of the auto-correlations for the respective disturbance vector x̅ₖ, given by

$${\sum }_{k = 1}^{n} {\overline{x}}_{k} \cdot {\overline{x}}_{k}^{T} .$$

This type of estimated covariance matrix Ĉ enables computation of a maximum of n (if n<m) or m (if m<n) eigenvectors having a respective length of m signal values. In an alternative implementation, the data matrix X is formed by arranging the disturbance vectors x̅ₖ as columns: X=[x̅₁ x̅₂ . . . x̅ₙ], where X has size m×n. The estimated covariance matrix C may still be given by ƒ(X^{T}·X) and has size n×n. This type of estimated covariance matrix C enables computation of a maximum of n (if n<m) or m (if m<n) eigenvectors having a respective length of n signal values. While the foregoing techniques of defining the estimated covariance matrix Ĉ are believed to be straightforward and sufficiently accurate, there are more advanced techniques that may be employed, including regularized or shrinkage estimators.

Returning to the flow chart in FIG. 2, step 203 may thus populate the covariance matrix Ĉ by estimated covariance values which are computed according to any of the techniques described in the foregoing, based on the set {x̅ₖ} provided by step 202.

Step 204 then computes eigenvectors and eigenvalues for the estimated covariance matrix Ĉ. The eigenvectors and eigenvalues may be computed using any known technique. In one example, step 204 computes the matrix V of eigenvectors which diagonalizes the estimated covariance matrix Ĉ:

V⁻¹·C·V=D,

where D is the diagonal matrix of eigenvalues of Ĉ. The column vectors of the matrix V represent the eigenvectors of the estimated covariance matrix Ĉ. The eigenvalues and eigenvectors are ordered and paired, i.e. the i:th eigenvalue in D corresponds to the i:th eigenvector in V. The computation of V and D typically involves the use of a dedicated computer-based algorithm for computing or numerically estimating eigenvectors and eigenvalues.

The principal components and variances may be directly given by the eigenvectors and eigenvalues. However, it is conceivable that the eigenvectors and eigenvalues are further processed by step 204 to produce the computed set of principal components and associated variances, e.g. by scaling the eigenvectors and/or the eigenvalues. In one further example, the set of eigenvectors are subjected to a transformation operation that makes the eigenvectors independent of each other, and the principal components are given by the resulting independent (and uncorrelated) eigenvectors. Such a transformation operation is well-known to the skilled person and makes sense to implement when the disturbance vectors x̅ₖ have a non-Gaussian distribution. The combination of PCA processing and the transformation operation is also known as Independent Component Analysis, ICA.

Step 205 then makes a selection among the principal components that were computed in step 204 to define a selected set of principal components. This selected set is designated by {ν̅ᵢ} in the following. The number of principal components to be selected in step 205 may be predefined. Typically, step 205 selects the principal components that are associated with the largest variance. The variance indicates the energy content in the respective principal component and thus its relative importance for the appearance of the disturbance vectors provided by step 202. It should be noted that if PCA processing is performed on an estimated correlation matrix Ĉ obtained from disturbance vectors x̅ₖ with non-zero mean, the mean is likely to be reflected in the most significant principal component(s). Such principal components may or may not be included among the principal components that are selected by step 205.

FIG. 3B and FIG. 3C are plots of the two most significant principal components ν̅₁, ν̅₂ resulting from PCA processing of the disturbance vectors x̅ₖ in FIG. 3A, which have been generated to have zero mean. The illustrated principal components ν̅₁, ν̅₂ have been normalized such that the length (|ν₁|, |ν̅₂|) of each principal component is 1. The length is equal to the square root of the sum of squares of the signal values in the principal component. As understood from the foregoing, first principal component ν̅₁ is computed to represent as much as possible of the variability among the disturbance vectors x̅ₖ, and the second principal component ν̅₂ is computed to be orthogonal to ν̅₁ and to represent as much as possible of the remaining variability among the disturbance vectors x̅ₖ.

If the process 200 is performed by the filtering device 9, step 205 may then store the selected set {ν̅ᵢ} of principal components in electronic memory 12 for use in subsequent filtering of the pressure signal P. Optionally, as described in further detail below, the principal components in the selected set {ν̅ᵢ} may be modified before being stored in memory 12. If the process 9 is performed on a separate device, the selected set {ν̅ᵢ} is instead transferred to the filtering device 9 for storage in its memory 12. The principal components stored in the memory 12 are hereinafter denoted a “current set of principal components” which is designated by {ν̅ᵢ}_{C}.

FIG. 2 is merely given as an example. The need to explicitly calculate (populate) the estimated covariance matrix Ĉ may be obviated, e.g. if the eigenvectors and eigenvalues are instead computed by the singular value decomposition (SVD) of X, as is well-known in the art. Thus, step 203 may be omitted. Further, the need to select a predefined number of principal components in step 205 may be obviated if step 204 outputs only this number of principal components, e.g. by step 204 being designed to only compute the most significant principal components, which thus form the select set {ν̅ᵢ}.

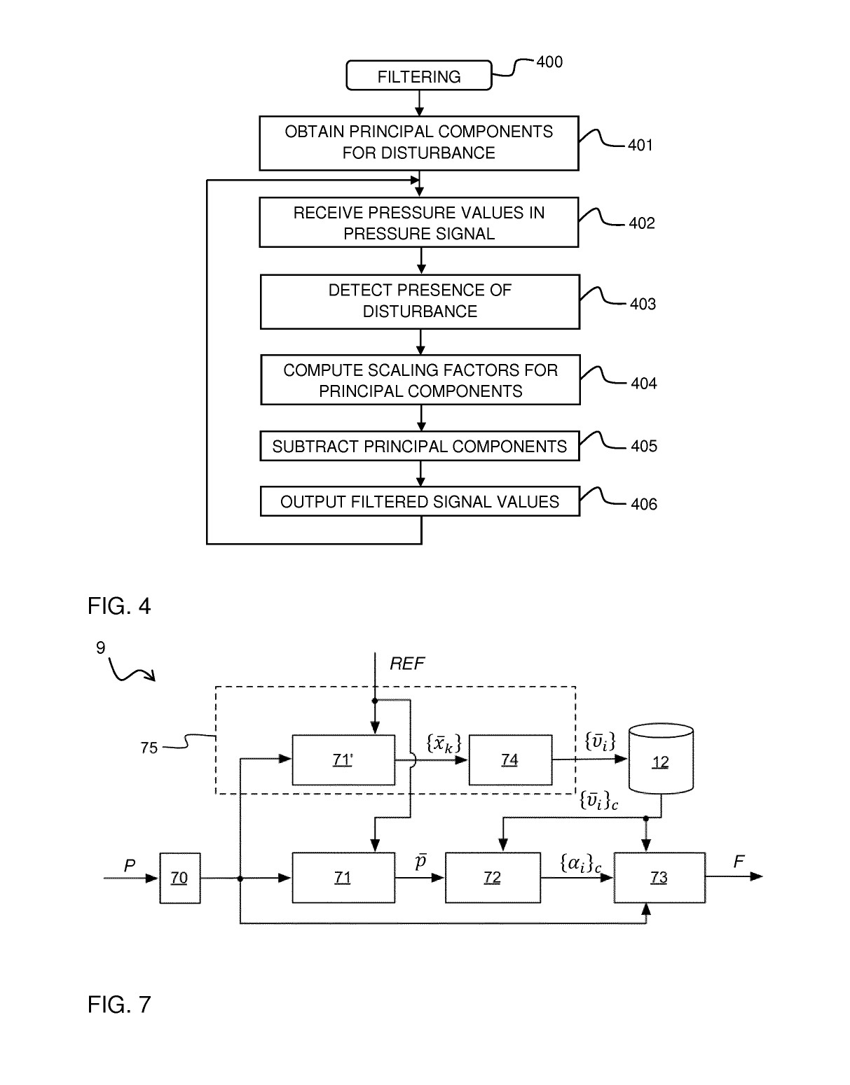

FIG. 4 illustrates a process 400 which is executed by the filtering device 9 of FIG. 1 for filtering the pressure signal P. Step 401 obtains a current set {ν̅ᵢ}_{C} of principal components for the disturbance to be suppressed in the pressure signal P. Step 401 may obtain the current set {ν̅ᵢ}_{C} by executing the process 200 in FIG. 2, e.g. as part of a start-up procedure in which the apparatus 1 prepares for a current treatment session. Alternatively, the current set {ν̅ᵢ}_{C} may previously have been computed separately from the device 9 and transferred to the device 9 for storage in memory 12. Thus, step 401 may obtain the current set {ν̅ᵢ}_{C} from memory 12. The process 400 then repeatedly executes steps 402-406 to generate a filtered signal in which the disturbance is suppressed. Step 402 receives pressure values of the pressure signal P, and step 403 detects presence of the disturbance in the pressure signal P and produces an estimated time point for each occurrence of the disturbance in the pressure signal P. Step 403 may operate to detect the presence of the disturbance in the same way as step 201, e.g. based on an external reference signal REF, which may be any type of signal provided by the apparatus 1 or sensors attached to or included in the apparatus 1. In one example, the reference signal REF is a control signal generated by a control unit in the apparatus 1. In another example, the reference signal REF is obtained from a sensor included in the disturbance sensor 7. In a further example, the reference signal REF is another pressure signal generated by a pressure sensor in the apparatus 1, such as the arterial sensor 8b or the TF sensor 8c. Alternatively, as explained for step 201, the time point may be estimated by processing the pressure signal P for detection of one or more dominating features of the disturbance. In one embodiment, the time point is estimated by calculating a cross-correlation between the first (most significant) principal component ν̅₁ and the pressure signal P, identifying the maximum correlation value resulting from the cross-correlation, and setting the estimated time point equal to the time point of the maximum correlation value.

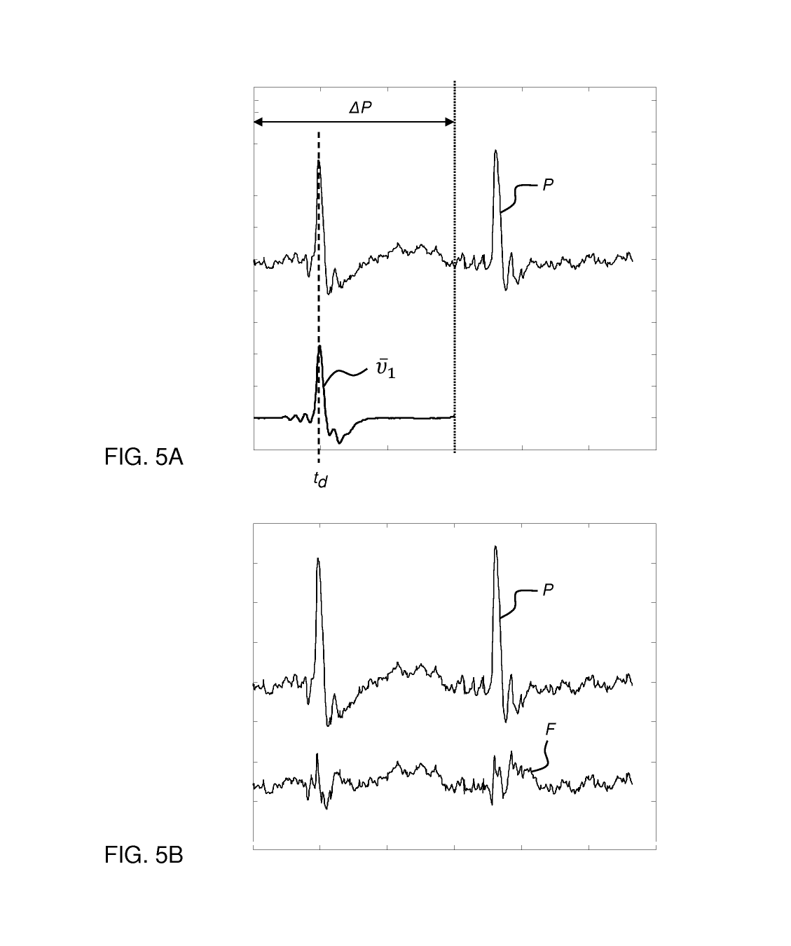

Step 404 computes scaling factors for the principal components ν̅ᵢ in the current set {ν̅ᵢ}_{C}, with respect to the disturbance identified in step 403. Based on the estimated time point obtained in step 403, a segment ΔP with the same length as the principal components ν̅ᵢ (containing m signal values) is selected in the pressure signal P. The segment ΔP is located around the disturbance in such a way that the estimated time point of the disturbance in the segment ΔP coincides with a corresponding time point in the principal components ν̅ᵢ. In other words, the segment ΔP is selected such that the location of the disturbance within the segment ΔP is the same as the location of the disturbance in the waveforms extracted in step 201. An example is shown in FIG. 5A, where the estimated time point t_{d} is given by the peak value of the disturbance in the pressure signal P and a segment ΔP is selected in the pressure signal P.

Then, a signal vector or pressure vector p̅ is populated with the signal values in the segment ΔP. For example, the pressure vector p̅ may be directly given by the segment:

$$\overline{p} = [ \begin{matrix}P ( 1 ) \\ P ( 2 ) \\ ⋮ \\ P ( m )\end{matrix} ]$$

where P(r) denotes the pressure value at position r within the segment ΔP. When the pressure vector p̅ has been obtained, step 404 calculates a weight or scaling factor αᵢ (i=1, 2, . . . , l) for each principal component ν̅ᵢ, where l is the number of principal components that are used in the filtering; l≧1. Thus, step 404 produces a current set of weights, designated herein by {αᵢ}_{C}. The weights αᵢ may be calculated as a scalar product of the respective principal component and the pressure vector: αᵢ=ν̅ᵢ^{T}·p̅, where ν̅ᵢ is given according to:

$${\overline{v}}_{i} = [ \begin{matrix}{v}_{i} ( 1 ) \\ {v}_{i} ( 2 ) \\ ⋮ \\ {v}_{i} ( m )\end{matrix} ] i = 1 , 2 , … , l$$

and νᵢ(r) denotes the signal value at position r within the principal component ν̅ᵢ In certain implementations, at least part of step 404 may be performed within step 403. If the estimated time point is computed by cross-correlating the most significant principal component ν̅₁ with the pressure signal P, the resulting maximum correlation value corresponds to the above-mentioned scalar product and is thus the weight α₁.

Step 405 subtracts the respective principal component ν̅ᵢ, scaled by the associated weight αᵢ, from the pressure vector p̅:

$$\overline{f} = \overline{p} - {\sum }_{i = 1}^{l} {a}_{i} {\overline{v}}_{i}$$

where ƒ̅ is a filtered vector consisting of filtered signal values for the time window ΔP. Reverting to FIG. 5A, step 405 corresponds to scaling the principal component ν̅₁ and then subtracting the thus-scaled principal component ν̅₁ from the pressure values within the segment ΔP in the pressure signal P.

Step 406 then outputs the filtered signal values. Step 406 may generate a filtered signal F by replacing the segment ΔP in the pressure signal P with the filtered signal vector ƒ̅. FIG. 5B illustrates such a filtered signal F generated by aligning and scaling the principal component ν̅₁ to each of the disturbances and subtracting the result from the pressure signal P.

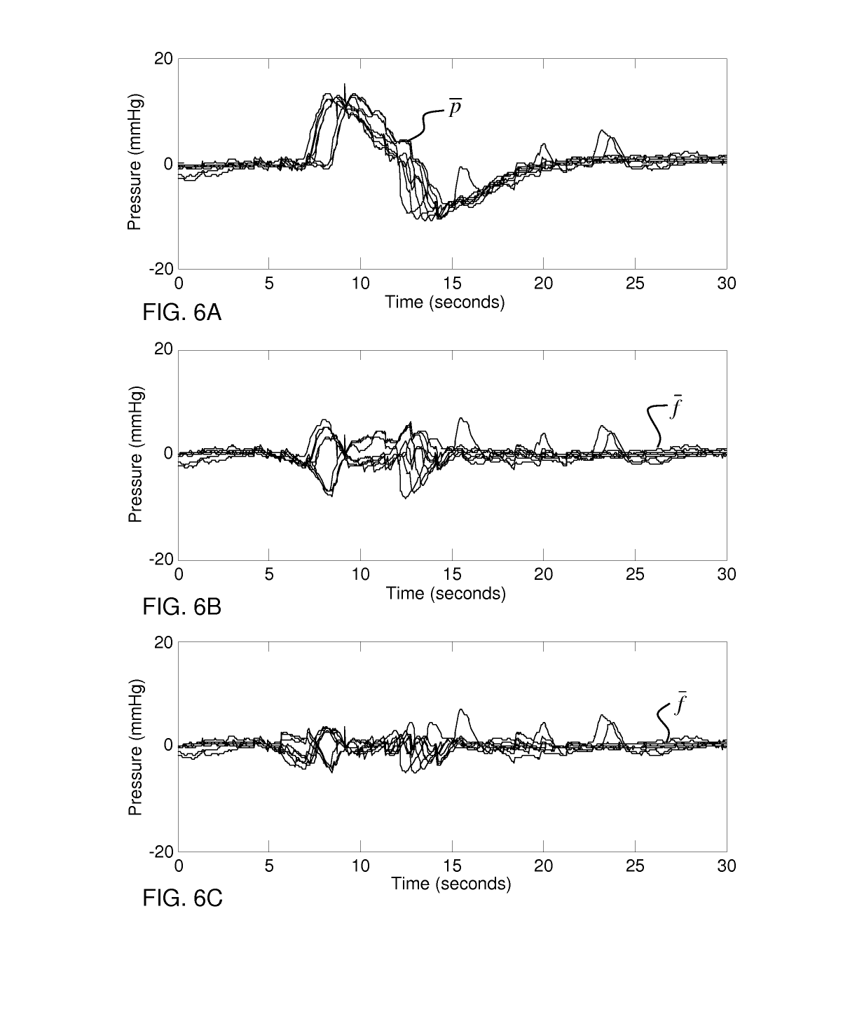

The operation of the process 400 is further exemplified in FIGS. 6A-6C. FIG. 6A illustrates a plurality of pressure vectors p̅ each representing a respective occurrence of the disturbance in the pressure signal P. FIG. 6B shows the corresponding filtered vectors ƒ̅ that are obtained after subtraction of the first principal component ν̅₁. FIG. 6C shows the corresponding filtered vectors ƒ̅ that are obtained after subtraction of both the first and second principal components ν̅₁, ν̅₂. As seen, the suppression of the disturbance is improved by the subtraction of two principal components. It is realized that further suppression may be achieved by subtracting further principal components. In certain implementations, it may be sufficient to subtract only the first principal component ν̅₁ from the pressure vectors p̅.

FIG. 7 is a block diagram of a structure for implementing the method of FIG. 4 in the filtering device 9. In the illustrated embodiment, the device 9 includes an electronic memory 12, a pre-processing block 70, disturbance detection blocks 71, 71′, a matching block 72, a subtraction block 73, and a PCA block 74. Although not shown, a control block may be provided to synchronize the operation of the blocks 70-74, and the blocks 70-74 may exchange data via the electronic memory 12.

The pre-processing block 70 is optional and may be configured to receive and pre-process the pressure signal P from the signal interface (10 in FIG. 1), e.g. for AD conversion, signal amplification, removal of offset, high frequency noise and supply voltage disturbances, etc. The removal of offset may be implemented to ensure that the pre-processed pressure signal has a zero mean, which may be advantageous for the subsequent PCA processing. As noted above, the pre-processing block 70 may also be configured to remove or suppress pump pulses and/or patient pulses in the incoming pressure signal P.

The disruption detection block 71, which implements step 403 in FIG. 4, is arranged to detect the disturbance in the (pre-processed) pressure signal P and populate a pressure vector p̅ for each disturbance. In the illustrated example, block 71 detects the disturbance based on the above-mentioned reference signal REF.

The matching block 72, which implements step 404 in FIG. 4, is configured to obtain the current set {ν̅ᵢ}_{C} of principal components from memory 12 and the pressure vector p̅ generated by block 71, and match the respective principal component to the pressure vector p̅ by computing a weight αᵢ for each principal component ν̅ᵢ. Block 72 thus provides a current set {αᵢ}_{C} of weights.

The subtraction block 73, which implements steps 405, 406 in FIG. 4, is configured to obtain the current sets {ν̅ᵢ}_{C}, {αᵢ}_{C} and generate the filtered signal vector ƒ̅. Block 73 is also configured to combine the (pre-processed) pressure signal P and the filtered signal vectors ƒ̅ into a filtered signal F.

The current set {ν̅ᵢ}_{C} of principal components used by blocks 72, 73 may be generated by the device 9, using a dedicated module 75 which is indicated by dashed lines in FIG. 7. The module 75 implements the process 200 in FIG. 2 and includes a detection block 71′ that implements steps 201-202 and a PCA block 74 that implements steps 203-205. Block 71′ may be identical to block 71, but populates disturbance vectors x̅ₖ instead of pressure vectors p̅. The module 75 generates the selected set {ν̅ᵢ} of principal components and stores it in memory 12 to form the current set {νᵢ}_{C} of principal components. In this process, the module 75 may replace (overwrite) any existing current set {ν̅ᵢ}_{C} already stored in memory 12. In one example, the module 75 is operated during manufacture or service to generate the selected set {ν̅ᵢ} which is then stored in memory 12 to define the current set {ν̅ᵢ}_{C}. In another example, the module 75 is operated during startup of a current treatment session, when a patient has been connected to the apparatus 1, to generate and store the principal components to be used for filtering during the current treatment session. The principal components are thus “treatment session specific”, in the sense that they are generated and used specifically for the current treatment session. In another example, the module 75 is operated before, during or after a current treatment session to generate principal components that are stored in memory 12 and used for filtering during the current treatment session and/or one or more forthcoming treatment sessions. In this example, the principal components may be generated to be either “treatment session specific”, “apparatus specific”, which means that they are generated and used specifically for the apparatus 1, or “apparatus-and-patient specific”, which means that they are generated and used for the specific combination of the apparatus 1 and the patient 100.

It should be understood that the module 75 may be omitted. In such an embodiment, the principal components may be generated separately from the device 9, using the process 200 in FIG. 2, and then transferred to the device 9 for storage in the memory 12. Such principal components are considered to be “pre-computed”. For example, the principal components may be stored in memory 12 during manufacture or service of the apparatus 1. Such principal components are typically either “apparatus specific” or “global”, which indicates that they are generic for all apparatuses of a specific type, e.g. by being generated based on disturbances recorded for a plurality of apparatuses of a specific type. It is also conceivable that the principal components are “patient specific”, which means that they are generated for a specific patient, e.g. based on disturbances recorded on different apparatuses of a specific type. Such “patient specific” principal components may be stored in the memory 12 in association with an identifier (“patient ID”) for the patient. The patient ID is input to the apparatus 1 or the device 9 when a treatment session is to be initiated for a specific patient, causing blocks 72, 73 to retrieve the “patient specific” principal components from memory 12.

FIG. 8 illustrates an alternative embodiment of the filtering device 9. For brevity of presentation, the following description will focus on differences compared to the embodiment in FIG. 7. Thus, the description of FIG. 8 is equally applicable to FIG. 7 unless otherwise stated. The embodiment in FIG. 8 includes an updating block 76, which is configured to modify the principal components that are stored in memory 12 as a function of the selected set {ν̅ᵢ} of principal components provided by PCA block 74. The updating block 76 may be operated to modify the stored principal components before, during or after a treatment session. This also means that the updating block 76 is operable to modify the current set {ν̅ᵢ}_{C} of principal components. Depending on implementation, the principal components that are modified by block 76 may be a set of pre-computed principal components, or a set of principal components that have previously been generated by the module 75 and stored in memory 12.

There are many different ways of modifying the stored principal components. For example, a newly computed principal component may modify a stored principal component in proportion to the number of disturbance vectors x̅ₖ, used for computing the stored principal component. For example, if the stored principal component is based on 1000 signal vectors x̅ₖ, the newly computed principal component is given a weight of 1/1000. Of course, other updating functions and weights may be used.

In variant (not shown), the updating block 76 may be configured to initiate a complete recalculation of the principal components based on the disturbance vectors x̅ₖ from a plurality of treatment sessions, provided that these disturbance vectors x̅ₖ, are stored in memory. In this variant, the updating block 76 may cause the PCA block 74 to retrieve the disturbance vectors x̅ₖ, from memory 12 and generate a selected set {ν̅ᵢ} of principal components. Thereby, the PCA block 74 may generate the principal components to represent a plurality of treatment sessions and possibly a plurality of patients. Thus, the selected set {ν̅ᵢ} may be either “apparatus specific” or “apparatus-and-patient specific”. The updating block 76 may then update the stored principal components based on the selected set {ν̅ᵢ}.

It is to be noted that the stored principal components may belong to any one of the above-identified categories. It is realized that the provision of the updating block 76 may improve the quality of the stored principal components, and thereby potentially improve the filtering performance of the device 9. For example, the updating enables “global” or “apparatus specific” principal components to be based on a larger set of disturbance vectors x̅ₖ, and the “apparatus-and-patient specific”, “patient specific” and “treatment session specific” principal components to be (gradually) adapted to the patient and the treatment session, respectively.

It should be understood that the appearance of the disturbance may change significantly during a treatment session, e.g. if the operation of the disturbance generator 7 is changed or if the operation or configuration of the apparatus 1 is changed so as to modify the dynamics of its hydraulic system, i.e. the EC circuit 1a and/or the TF circuit 1b. Some non-limiting examples include a change of dialyzer 5, a change of pressure in a venous drip chamber in the EC circuit 1a, a change of treatment fluid temperature, and a change in treatment fluid flow rate. The filtering device 9 in FIG. 8 may automatically account for such changes in appearance of the disturbance, if the module 75 is operated to continuously or intermittently modify the current set {ν̅ᵢ}_{C} of principal components. Alternatively, the filtering device 9 may be triggered to update the current set of principal components following a change in the operation or configuration of the apparatus 1 (including the disturbance generator 7). In FIG. 8, the module 75 may be triggered to modify the current set {ν̅ᵢ}_{C} of principal components stored in memory 12. In FIG. 7, the module 75 may be triggered to generate and store, in memory 12, a new set of principal components to form the current set {ν̅ᵢ}_{C} of principal components.

The filtering device 9 may be pre-configured to operate with principal components according to only one of the categories described in the foregoing, i.e. “global”, “apparatus specific”, “apparatus-and-patient specific”, “patient specific” or “treatment session specific”. Alternatively, the filtering device 9 may allow an operator to select, via a user interface (not shown) such as a touch screen, keyboard, keypad, etc, a category to be used when filtering the pressure signal P. For example, the device 9 may store sets of principal components for different categories in memory 12 and be configured to instruct the blocks 72, 73 to retrieve the current set {ν̅ᵢ}_{C} of principal components based on the operator's selection of category. In a further alternative, the device 9 is operable to monitor the quality of the filtered signal F and switch, based on quality, between different categories of principal components and/or change the number of principal components that are subtracted from the pressure vectors. The quality may be estimated by measuring the variability of the filtered signal F during a test period.

Going from principal components that are “global” or “apparatus specific” to principal components that are “apparatus-and-patient specific”, “patient specific” or “treatment session specific” may increase the accuracy of the filtered signal F and/or enable the use of fewer principal components, since the principal components are more likely to be tailored to the actual appearance of the disturbances in the pressure signal P to be filtered. On the other hand, the use of principal components that are “global” or “apparatus specific” may reduce the complexity and processing requirement of the device 9. Further, the quality of the principal components also depends on the number of disturbance vectors x̅ₖ, that are input to PCA block 74. Thus, it may be preferable to generate and use “global” or “apparatus specific” principal components if this is believed to increase the quality.

To exemplify the potential difference between principal components of different categories, FIG. 9A is a plot of a plurality of first principal components ν̅₁ (solid lines) computed by block 74 in FIG. 7 based on a set of disturbance vectors acquired during a respective treatment session. The illustrated first principal components ν̅₁ are all computed for one and the same apparatus 1. In this example, there are discernible variations in shape between the “treatment session specific” principal components ν̅₁. FIG. 9A also illustrates the corresponding “apparatus specific” principal component ν̅₁ (dotted line) that is generated by block 74 when operating on the ensemble of disturbance vectors that are used for generating the “treatment session specific” principal components in FIG. 9A. FIG. 9B illustrates corresponding results for the second principal component ν̅₂ computed by block 74.

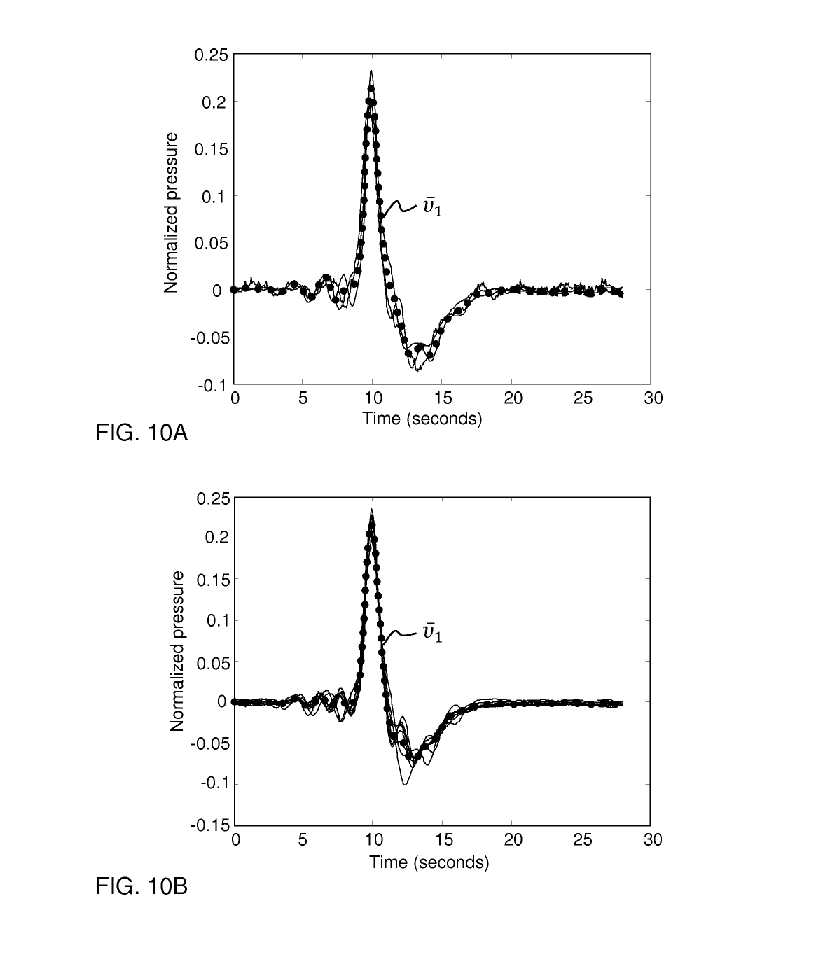

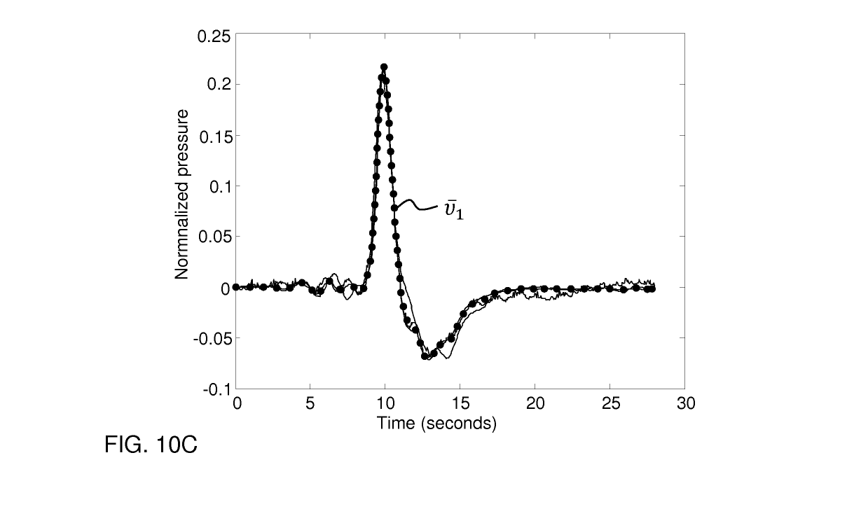

FIG. 10A corresponds to FIG. 9A but illustrates “treatment session specific” principal components ν̅₁ (solid lines) and an “apparatus specific” principal component ν̅₁ (dotted line) which are generated to represent another disturbance in the pressure signal P. FIG. 10B illustrate first principal components ν̅₁ generated for another apparatus of the same type as the apparatus in FIG. 10A. A comparison of FIG. 10A and FIG. 10B reveals a discernible difference in shape between the “apparatus specific” principal components. To further exemplify such difference, FIG. 10C illustrates three “apparatus specific” principal components ν̅₁ (solid lines) generated for three different apparatuses. FIG. 10C also illustrates the corresponding “global” principal component ν̅₁ (dotted line) that is generated by block 74 when operating on the ensemble of disturbance vectors that are used for generating the “apparatus specific” principal components in FIG. 10C.

As noted above, the appearance of the disturbance may change significantly with the operating condition of the apparatus 1. The operating condition is given by settings. As also indicated above, settings with potential impact on the disturbance include the pressure in the venous drip chamber, the treatment fluid temperature, and the treatment fluid flow rate. It is conceivable that the memory 12 stores different sets of principal components in association with different settings or combinations of settings. Blocks 72, 73 (FIGS. 7-8) may be configured to retrieve the current set {ν̅ᵢ}_{C} of principal components from the memory 12 based on a current setting or combination of settings for the apparatus 1. Alternatively or additionally, blocks 72, 73 may obtain the current set {ν̅ᵢ}_{C} of principal components by use of a functional relation that operates on one or more sets of principal components, which are retrieved from the memory 12 based on the current setting or combination of settings. Such a functional relation may involve interpolation, extrapolation, scaling in time or scaling in amplitude. Although not shown in FIGS. 7-8, a dedicated block may be provided to retrieve data from the memory 12, apply the functional relation and provide the current set {ν̅ᵢ}_{C} of principal components to the blocks 72, 73.

The filtering device 9 may be implemented by special-purpose software (or firmware) run on one or more general-purpose or special-purpose computing devices. In this context, it is to be understood that an “element” or “means” of such a computing device refers to a conceptual equivalent of a method step; there is not always a one-to-one correspondence between elements/means and particular pieces of hardware or software routines. One piece of hardware sometimes comprises different means/elements. For example, a processing unit serves as one element/means when executing one instruction, but serves as another element/means when executing another instruction. In addition, one element/means may be implemented by one instruction in some cases, but by a plurality of instructions in some other cases. Such a software controlled computing device may include one or more processing units (cf. 11 in FIG. 1), e.g. a CPU (“Central Processing Unit”), a DSP (“Digital Signal Processor”), an ASIC (“Application-Specific Integrated Circuit”), discrete analog and/or digital components, or some other programmable logical device, such as an FPGA (“Field Programmable Gate Array”). The device 9 may further include a system memory and a system bus that couples various system components including the system memory (cf. 12 in FIG. 1) to the processing unit. The system bus may be any of several types of bus structures including a memory bus or memory controller, a peripheral bus, and a local bus using any of a variety of bus architectures. The system memory may include computer storage media in the form of volatile and/or non-volatile memory such as read only memory (ROM), random access memory (RAM) and flash memory. The special-purpose software may be stored in the system memory, or on other removable/non-removable volatile/non-volatile computer storage media which is included in or accessible to the computing device, such as magnetic media, optical media, flash memory cards, digital tape, solid state RAM, solid state ROM, etc. The device 9 may include one or more communication interfaces (cf. 10 in FIG. 1), such as a serial interface, a parallel interface, a USB interface, a wireless interface, a network adapter, etc, as well as one or more data acquisition devices, such as an A/D converter. The special-purpose software may be provided to the device 9 on any suitable computer-readable medium, including a record medium or a read-only memory.

It is also conceivable that some (or all) elements/means are fully or partially implemented by dedicated hardware, such as an FPGA, an ASIC, or an assembly of discrete electronic components (resistors, capacitors, operational amplifier, transistors, filters, etc), as is well-known in the art.

It should be emphasized that the invention is not limited to digital signal processing, but could be fully implemented by a combination of analog devices.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

The inventive technique is applicable for filtering in all types of EC circuits in which blood is taken from the systemic blood circuit of the patient to interact with a treatment fluid in a blood processing unit and is then returned to the patient. Such blood flow circuits include circuits for hemodialysis, hemofiltration, hemodiafiltration, continuous renal replacement therapy, extracorporeal liver support/dialysis, and heart congestion failure treatment. The extracorporeal blood flow circuit may be connected to the patient by separate access devices for blood removal and blood return, or by a common access device (“single-needle”).

It is to be understood that the inventive filtering technique may be applied to suppress several different disturbances in the pressure signal, e.g. disturbances of different origin and different appearance. A respective set of principal components may be computed for each different disturbance, using the process 200 in FIG. 2, and be applied for suppression of the respective disturbance, using the process in FIG. 4.

The inventive technique need not operate on real-time data, but could be used for processing off-line data, such as a previously recorded pressure signal.

1: A signal filtering device comprising:
an input for receiving a pressure signal from a pressure sensor, which is arranged in a blood processing apparatus and is responsive to pressure variations in blood that is pumped in an extracorporeal blood circuit for passage through a blood processing unit, at least part of the pressure variations originating from a known disturbance generator in the blood processing apparatus and resulting in a disturbance in the pressure signal,
a signal processor connected to the input and being configured to, in connection with a current session of the blood processing apparatus:
obtain a current set of principal components representing the disturbance, the respective principal component in the current set being generated by Principal Component Analysis, PCA, of a plurality of characteristic waveforms representing the disturbance,
detect presence in the pressure signal of a current disturbance that originates from the known disturbance generator,
compute a scaling factor for the respective principal component with respect to the current disturbance, and
subtracting the respective principal component, scaled in magnitude by the respective scaling factor, from the pressure signal.

2: The signal filtering device of claim 1, wherein the current set consists of at least two principal components.

3: The signal filtering device of claim 1, wherein the signal processor is configured to:
extract all or part of the characteristic waveforms from the pressure signal, each characteristic waveform representing a respective occurrence of the disturbance in the pressure signal;
process all or part of the characteristic waveforms by PCA so as to compute a set of principal components for all or part of the characteristic waveforms;
define a selected set of principal components among the set of principal components; and
generate the current set of principal components as a function of the selected set of principal components.

4: The signal filtering device of claim 3, wherein the signal processor is configured to, when processing all or part of the characteristic waveforms by PCA, compute a corresponding set of variances for the set of principal components, and to select, based on the set of variances, the principal components for the selected set of principal components.

5: The signal filtering device of claim 3, wherein the signal processor is configured to, when processing all or part of the characteristic waveforms by PCA, to compute a set of eigenvectors for an estimated covariance matrix with estimated covariance values for all or part of the characteristic waveforms, the set of principal components being given by the set of eigenvectors.

6: The signal filtering device of claim 5, wherein the estimated covariance matrix is given by ƒ(X^{T}X), wherein X is a matrix with all or part of the characteristic waveforms arranged as rows or columns, X^{T} is a transpose of the matrix X, and ƒ is a linear function.

7: The signal filtering device of claim 5, wherein the signal processor is configured, when processing all or part of the characteristic waveforms by PCA, to: compute the estimated covariance values; populate the estimated covariance matrix by the estimated covariance values; and process the estimated covariance matrix for computation of the set of eigenvectors.

8: The signal filtering device of claim 3, wherein the signal processor is configured to extract all or part of the characteristic waveforms from the pressure signal during the current session, such as during a startup phase of the current session.

9: The signal filtering device of claim 3, wherein the signal processor is configured to use the selected set of principal components as the current set of principal components.

10: The signal filtering device of claim 3, wherein the signal processor is configured to: retrieve a stored set of principal components from an electronic memory associated with the signal filtering device; and obtain the current set of principal components as a combination of the stored set of principal components and the selected set of principal components.

11: The signal filtering device of claim 10, wherein the signal processor is configured to: store the current set of principal components in the electronic memory so as to replace the stored set of principal components in the electronic memory.

12: The signal filtering device of claim 10, wherein the stored set of principal components are pre-computed externally of the signal filtering device.

13: The signal filtering device of claim 1, wherein the signal processor is configured to obtain the current set of principal components by retrieving a pre-computed set of principal components from an electronic memory associated with the signal filtering device, the pre-computed set of principal components being generated externally of the signal filtering device.

14: The signal filtering device of claim 13, wherein the pre-computed set of principal components is specific to one of: an apparatus type of the blood processing apparatus, the blood processing apparatus, a patient connected to the blood processing apparatus, and a combination of the blood processing apparatus and the patient.

15: The signal filtering device of claim 1, wherein the signal processor is operable to obtain the current set of principal components to be specific to one of: an apparatus type of the blood processing apparatus, the blood processing apparatus, a patient connected to the blood processing apparatus, a combination of the blood processing apparatus and the patient, and the current session.

16: The signal filtering device of claim 1, wherein the signal processor is configured to: compute the scaling factor as a scalar product of the respective principal component in the current set of principal components and a signal vector representing the current disturbance in the pressure signal.

17: The signal filtering device of claim 1, wherein the signal processor is configured to detect the presence of the current disturbance by one or more of:
processing the pressure signal for detection of a dominant feature of the disturbance;
cross-correlating a selected principal component in the current set of principal components with the pressure signal to generate a plurality of correlation values, and processing the plurality of correlation values for detection of the current disturbance; and
receiving, via the input, a reference signal which is indicative of the operation of the known disturbance generator, and detecting, based on the reference signal, an activation or deactivation of the known disturbance generator that results in the current disturbance.

18: The signal filtering device of claim 1, wherein the signal processor is configured to: determine a time point of the current disturbance in the pressure signal; and align the respective principal component with respect to the time point when subtracting the respective principal component from the pressure signal.

19: The signal filtering device of claim 1, wherein the signal processor is further configured to: generate a filtered signal, and process the filtered signal for detection of pulsations originating from one or more physiological pulse generators in the patient.

20: A signal filtering device comprising:
an input module for receiving a pressure signal from a pressure sensor, which is arranged in a blood processing apparatus and is responsive to pressure variations in blood that is pumped in an extracorporeal blood circuit for passage through a blood processing unit, at least part of the pressure variations originating from a known disturbance generator in the blood processing apparatus and resulting in a disturbance in the pressure signal,
a memory module for providing a current set of principal components representing the disturbance, the respective principal component in the current set of principal components being generated by Principal Component Analysis, PCA, of a plurality of characteristic waveforms representing the disturbance in the pressure signal,
a detection module for detecting presence in the pressure signal of a current disturbance that originates from the known disturbance generator,
a computing module for computing a scaling factor for the respective principal component with respect to the current disturbance, and
a subtraction module for subtracting the respective principal component, scaled in magnitude by the respective scaling factor, from the pressure signal.

21: A method of filtering, the method comprising:
acquiring a pressure signal originating from a pressure sensor, which is arranged in a blood processing apparatus and is responsive to pressure variations in blood that is pumped in an extracorporeal blood circuit for passage through a blood processing unit, at least part of the pressure variations originating from a known disturbance generator in the blood processing apparatus and resulting in a disturbance in the pressure signal,
obtaining a current set of principal components representing the disturbance, the respective principal component in the current set of principal components being generated by Principal Component Analysis, PCA, of a plurality of characteristic waveforms representing the disturbance in the pressure signal,
detecting presence in the pressure signal of a current disturbance that originates from the known disturbance generator,
computing a scaling factor for the respective principal component with respect to the current disturbance, and
subtracting the respective principal component, scaled in magnitude by the respective scaling factor, from the pressure signal.

22: A computer-readable medium comprising computer instructions which, when executed by a processor, cause the processor to perform the method of claim 21.

23: A blood processing apparatus comprising:
an extracorporeal blood circuit;
a pressure sensor, the pressure sensor being responsive to pressure variations in blood that is pumped in the extracorporeal blood circuit for passage through a blood processing unit; and
the signal filtering device of claim 1 which is connected to receive a pressure signal from the pressure sensor.
